(19) United States
(12) Patent Application Publication
(10) Pub. No.: US 2012/0022583 A1
(43) Pub. Date: Jan. 26, 2012

(54) Surgical Tool with Crossbar Lever

(76) Inventors: Eric Sugalski, Cambridge, MA (US); Erik Holverson, Naperville, IL (US); Christopher Houghton, Chicago, IL (US)

(21) Appl. No.: 12/842,399

(22) Filed: Jul. 23, 2010

(51) Int. Cl. A61B 17/28

(52) U.S. Cl. 606/205

(57) ABSTRACT

A crossbar lever of a surgical tool has a bridge portion which extends forward and a transverse portion. The bridge portion is retained between the second and third fingers of the surgeon's hand, and this retention establishes greater control and stability of the surgical tool. The crossbar lever pivots relative to a handgrip of a handle assembly and a shaft assembly extends from the handle assembly. Tissue contacting or manipulating devices such as jaws are located at the forward end of the shaft assembly. The surgical tool is particularly useful in performing minimally invasive surgical procedures.

### CROSS REFERENCE TO RELATED INVENTIONS

This application is related to U.S. patent application Ser. No. (24.373), titled Jaw Movement Mechanism and Method for Surgical Tool, and to U.S. patent application Ser. No. (24.376), titled Tissue Fusion System and Method for Performing a Self Test, and to U.S. patent application Ser. No. (24.377), titled Tissue Fusion System and Method of Performing a Functional Verification Test, all of which were filed concurrently herewith and assigned to the assignee hereof. The subject matter of these related patent applications are incorporated herein by this reference.

### FIELD OF THE INVENTION

This invention relates to surgical tools used to grasp or compress tissue between a pair of jaws. More particularly, the present invention relates to a new and improved surgical tool having a crossbar lever which permits greater surgeon control during use of the surgical tool.

### BACKGROUND OF THE INVENTION

Specialized surgical tools are required for many types of modern medical procedures, particularly minimally invasive surgical procedures. Minimally invasive surgery uses surgical tools having jaws or other tissue contacting and manipulating devices located at the end of a long and narrow shaft assembly. A handle assembly operating mechanism is connected at the other end of the shaft to operate the jaws or tissue-contacting devices. The long shaft assembly permits the jaws or tissue-contacting devices to be extended to the surgical site within the patient's body while the surgeon grasps and manipulates the surgical tool using the handle assembly at a location outside of the patient's body.

Minimally invasive laparoscopic surgery typically involves making a few small incisions through the outer muscular wall of the body, inserting cannulas through the incisions, adding carbon dioxide or argon gas to inflate the body wall away from the internal organs and thereby create a body cavity, inserting a miniature camera, a light and the surgical tools through working channels of the cannulas into the body cavity, and performing the surgical procedure using the surgical tools with the aid of the camera while the gas maintains the inflated condition of the body cavity. Minimally invasive endoscopic surgery typically involves inserting an endoscope through an orifice of the body to gain access to an internal organ such as the lungs, stomach or intestines, and inserting a surgical tool through a working channel of the endoscope. In some circumstances another working channel of the endoscope houses a camera or other optical viewing device and light. Other types of surgical tools require the jaws or other tissue-contacting devices to contact and manipulate the tissue.

One type of frequently-performed minimally invasive procedure is coaptive thermal tissue sealing or fusion. Coaptive thermal tissue sealing involves the application of force and thermal energy to compress and heat tissue sufficiently to join together separate pieces of tissue. The tissue is typically fused or sealed to prevent blood or other fluid loss. Coaptive tissue sealing avoids the need to manually surture or tie-off vessels during a surgical procedure, which would be very difficult to perform in a minimally invasive procedure. Sealing the tissue allows the tissue to be cut adjacent to the fused area without blood or fluid loss.

Sealing blood vessels is of particular concern, because a failed vessel seal after the conclusion of surgery leads to internal bleeding. Internal bleeding usually requires a second operation to gain access to and seal the leaking vessel, which induces further trauma and risk to the patient.

Modern tissue sealing tools heat the tissue by passing a radio frequency (RF) current through the tissue or by applying thermal energy to the tissue from heating elements. In both cases, the jaws at a forward or front end of the shaft assembly grasp and compress the tissue and also deliver energy to the tissue. In RF tissue sealing tools, the jaws also function as electrodes which conduct the RF current through the tissue grasped between the jaws. In thermal heating tissue sealing tools, the heating element is incorporated with the jaws to transfer the thermal energy to the tissue grasped between the jaws.

A relatively large amount of compressive force must be applied to the tissue from the jaws while the tissue is heated to achieve an adequate seal. The jaws open and close as a result of pivoting movement of a lever with respect to a handgrip the handle assembly. A surgeon grasps the handgrip in one hand, and the fingers of that hand squeeze the lever and pivot it toward the handgrip. An operating mechanism of the handle assembly converts the pivoting action of the lever into mechanical force and movement which is transferred through the shaft assembly to the jaws, causing the jaws to move relative to one another. Because more force is typically required to close the jaws and compress the tissue than is required to open the jaws and release the tissue, and because human hand strength is much greater when squeezing the fingers rather than opening the fingers, the operating mechanism of the handgrip assembly and the jaws are interconnected to close the jaws when the lever is pivoted toward the handgrip.

The typical lever configuration is oval shaped. The surgeon inserts his or her fingers through the oval-shaped opening. The oval-shaped lever has the advantages that the rear portion of the oval-shaped lever closest to the handgrip may be squeezed and pivoted toward the handgrip to close the jaws, while the front portion of the oval-shaped lever which extends in front of the fingers is available to be contacted by opening movement of the fingers to assist in opening the jaws. Since the oval-shaped configuration surrounds the fingers, the surgeon may more easily rotate the entire surgical tool by a combination of finger and wrist movement. However, since the handgrip is not entirely enclosed or surrounded by the hand and the fingers which extend to the lever, the handle assembly is somewhat loosely positioned in the surgeon's hand until the lever is grasped and pivoted. Manipulating the surgical tool while the handle assembly is loosely positioned in the surgeon's hand with the fingers extended forward is facilitated by the ability to contact both the front and rear parts of the oval-shaped lever. Contacting both the front and the rear parts of the ovals-shaped lever with the extended fingers facilitates stabilizing the tool while it is oriented in a desired position by the physician.

### SUMMARY OF THE INVENTION

The present invention creates an enhanced degree and ease of control over the manipulation of a surgical tool of the type which has a lever which is pivoted relative to a handgrip of a handle assembly. The present invention involves the use of a lever which has a T-shaped crossbar extending at the front of the lever. The surgeon extends his or her middle two fingers on opposite sides of a bridge or leg portion of the T-shaped crossbar. Locating the two middle fingers on opposite sides of the bridge portion of the crossbar allows the surgeon to apply positive firm contact with the lever and achieve a greater degree of precision in manipulation than is possible when all four fingers of the surgeon's fingers are located within the center of the relatively large oval opening prior art lever. The better contact with the lever also stabilizes the surgical tool against inadvertent movement. A foreword transverse portion of the T-shaped lever is available for the surgeon to contact with opening finger movement in order to assist in moving the jaws away from one another. The forward transverse portion of the T-shaped lever is also available to give the surgeon the opportunity to change where on the crossbar lever the rearward squeezing force is applied by the fingers, when it is desired to apply a greater amount of squeezing force to compress the tissue between the jaws. The T-shaped crossbar lever also offers an ideal location for locating control buttons and switches for easy contact by the fingers to control the delivery of electrical energy during use of the tool.

One aspect of the invention relates to an improved surgical tool having a handle assembly which includes a handgrip and a lever which pivots relative to the handgrip in response to squeezing movement from fingers of a user's hand that also contacts the handgrip. An elongated shaft assembly is connected to and extends from the handle assembly. Tissue contact elements, for example jaws, are connected to a forward end of the shaft assembly for manipulating tissue. An operating mechanism within the handle assembly transfers pivotal movement of the lever relative to the handgrip into motion which moves the tissue contact elements. The lever is a crossbar lever having a base portion and a bridge portion and a transverse portion configured generally in an H-shape. The transverse portion is located forward of the base portion and extends generally in alignment with the base portion. The bridge portion extends from a general midpoint of the transverse portion rearward to the base portion. The H-shape configuration of the base, bridge and transverse portions define an upward facing U-shaped opening for receiving first and second fingers of the user's hand and a downward facing U-shaped opening for receiving third and fourth fingers of the user's hand. The bridge portion extends between the second and third fingers of the user's hand and receives squeezing force from the second and third fingers to stabilize and control the surgical tool in the hand of the user.

Subsidiary features of this aspect of the invention include one of the base portion or the transverse portion receiving squeezing force from the fingers to pivot the crossbar lever toward the handgrip, a control element such as electrical switch connected to the transverse portion at a location to be contacted by one of the fingers to control an aspect of surgical operation of the tool while the other fingers squeeze the one of the base portion or the transverse portion, a flange extending on opposite lateral sides and around the rear portion of the handgrip at a location vertically adjacent to the handgrip to be contacted by a thumb and a joint of a first finger of the hand to further control and stabilize the surgical tool, and a control element such as electrical switch connected to the flange at a location to be contacted by the thumb to control an aspect of surgical operation of the tool.

Another aspect of the invention relates to the improved surgical tool in which the operating mechanism comprises a rocker arm connected with the lever to pivot in response to pivoting movement of the lever, an adapter connected to one of the shaft members and contacted by the rocker arm to transfer pivoting movement of the rocker arm through the adapter to one of the shaft members as longitudinally reciprocating motion, a link member pivotally connecting the crossbar lever to the rocker arm to transfer force from the crossbar lever to the rocker arm as the crossbar lever is pivoted, and a force transfer mechanism connected between the adapter and the one of the shaft members. The force transfer mechanism includes a cap member connected to the one shaft member and a spring which surrounds the one shaft member and which extends between the adapter and the cap member. The spring limits the amount of force transferred between the adapter and the one shaft member.

Subsidiary features of this aspect of the invention relate to a cap member connected to the one shaft member and a spring which surrounds the one shaft member and extends between the adapter and the cap member, precompressing the spring to a predetermined extent between the adapter and the cap member so that the spring accepts additional force beyond the predetermined extent of precompression arising from force transferred from the rocker arm to the adapter upon pivoting of the crossbar lever, defining annular openings in both the rocker arm and the crossbar lever and extending the force transfer mechanism and the one shaft member extend through the annular openings of both the rocker arm and the crossbar lever and selectively rotating the shaft assembly relative to the handle assembly.

Other aspects and features of the invention, as well as a more complete understanding of the present invention and its scope may be obtained from the accompanying drawings, which are briefly summarized below, from the following description of a presently preferred embodiment of the invention, and from the appended claims.

### BRIEF DESCRIPTION OF THE DRAWINGS

FIG. 1 is a perspective view of a surgical tool having a crossbar lever which incorporates the present invention.

FIG. 2 is a side elevation view of the surgical tool shown in FIG. 1, illustrating squeezing force applied to the crossbar lever to close jaws of the surgical tool.

FIG. 3 is a side elevation view of the surgical tool shown in FIGS. 1 and 2, illustrating an alternative application of squeezing force to the crossbar lever compared to that application shown in FIG. 2.

FIG. 4 is a side elevation view of the surgical tool shown in FIGS. 1-3, illustrating the application of forward finger movement to the crossbar lever to assist in opening the jaws.

FIG. 5 is a side elevation view of the surgical tool shown in FIGS. 1-4, illustrating contact of a control button on the crossbar lever by a finger of the surgeon.

FIG. 6 is a partially sectioned elevational view of the surgical tool shown in FIGS. 1 and 4, showing components of an operating mechanism located within a handle assembly when the crossbar lever is pivoted to a position which opens the jaws.

FIG. 7 is a view of the surgical tool similar to FIG. 6, showing components of the operating mechanism when the crossbar lever is pivoted to a position which closes the jaws.

FIG. 8 is a perspective view of components of the operating mechanism of the surgical tool shown in FIGS. 6 and 7, separated from the handle assembly and interacting with a shaft assembly, a jaw movement mechanism and the jaws of the surgical tool.

FIG. 9 is a side elevation view of a force limitation mechanism of the surgical tool shown in FIGS. 6-8, illustrating a force limiting spring in a pre-compressed state.

FIG. 10 is a side elevation view of the force limitation mechanism shown in FIG. 9, illustrating the force limitation spring compressed further than the pre-compressed state shown in FIG. 9.

FIG. 11 is an exploded perspective view of the force limitation mechanism shown in FIGS. 9 and 10.

FIG. 12 is a perspective view of a spinner of the surgical tool shown in FIGS. 1-5.

FIG. 13 is an elevation view of components of the operating mechanism located in the rear of the housing of the surgical tool shown in FIGS. 7 and 8.

### DETAILED DESCRIPTION

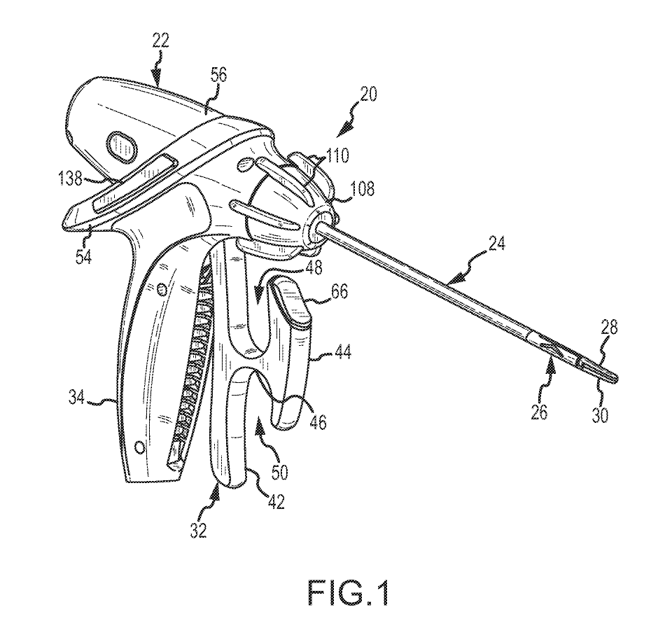

A surgical tool 20 which is useful in performing minimally invasive surgical procedures and which incorporates the present invention, is shown in FIG. 1. The surgical tool 20 includes a handle assembly 22, a shaft assembly 24 connected to the handle assembly, a jaw movement mechanism 26 located at a forward or front end of the shaft assembly 24, and jaws 28 and 30 connected to and moved by the jaw movement mechanism 26.

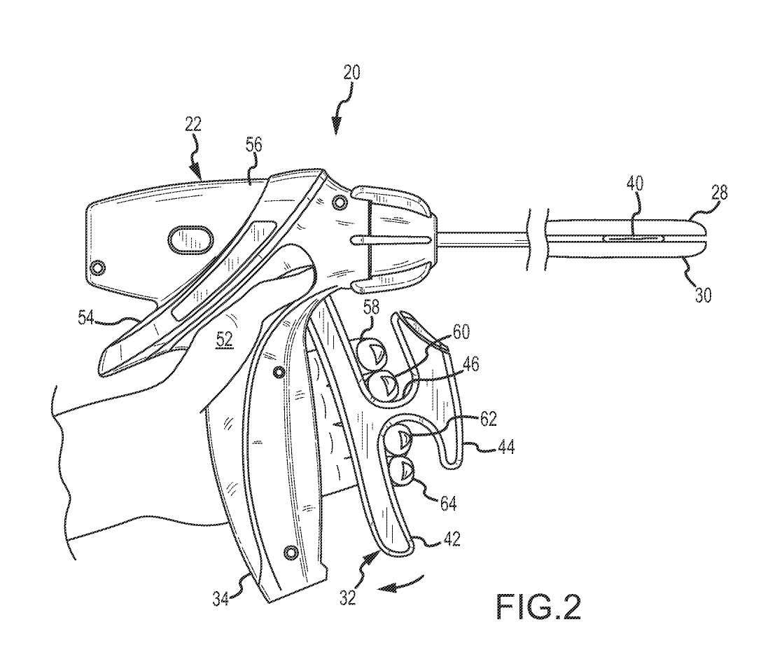

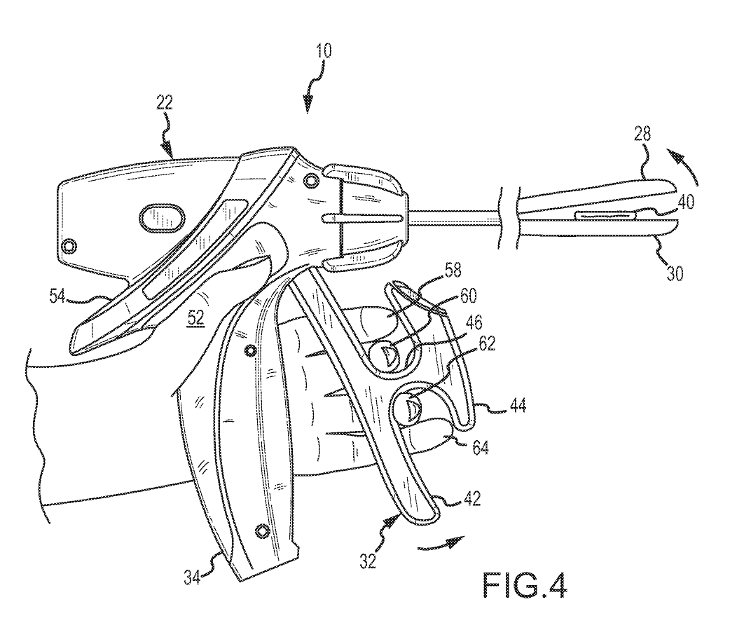

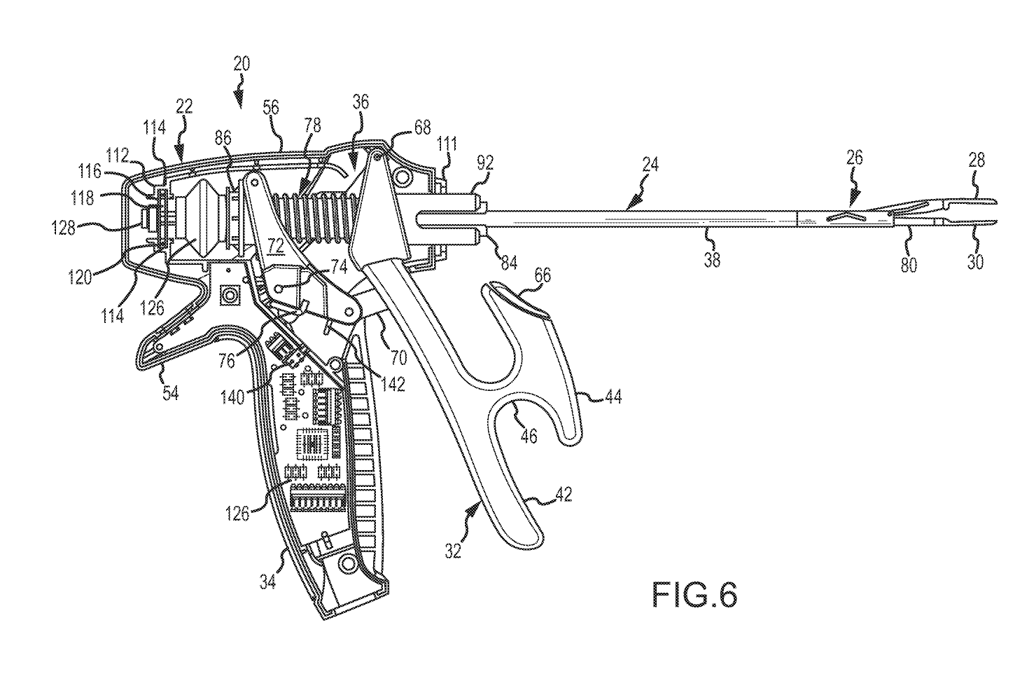

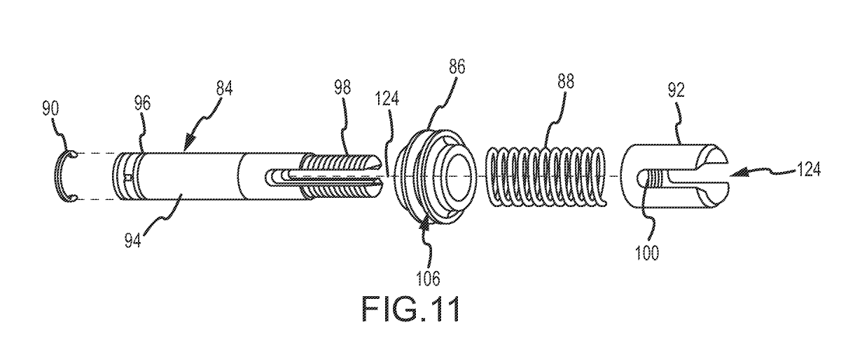

A movable crossbar lever 32 of the handle assembly 22 pivots relative to a fixed handgrip 34, as shown in FIGS. 2-4. The fixed handgrip 34 is contacted by the heel and thumb of the surgeon's hand, and the four fingers of the surgeon's hand extend to contact and squeeze the crossbar lever 32. An internal operating mechanism 36 (FIGS. 6-8) within the handle assembly 22 converts the relative pivoting movement of the lever 32 and handgrip 34 into relative longitudinal reciprocating movement of relatively movable shaft members of the shaft assembly 24. The longitudinally relatively movable shaft members are a sleeve housing 38 and an interior stationary rod (80, FIG. 11) within the sleeve housing 38. The jaw movement mechanism 16 converts the relative longitudinal reciprocating motion of the shaft members into movement of the jaws 28 and 30 into and between a closed position (FIGS. 2, 3, 5, 7) and an open position (FIGS. 4 and 6). In the closed position, the jaws 28 and 30 capture and compress tissue 40 between them. One example of a jaw movement mechanism is described in the above-referenced U.S. patent application Ser. No. (24.373).

A significant feature of the present invention is the crossbar lever 32. The crossbar lever 32 includes a base portion 42, a transverse portion 44 and a bridge portion 46 which connects the base portion 42 to the transverse portion 44. The bridge portion 46 connects with the transverse portion 44 at approximately a midpoint along the length of the transverse portion. The shape of the crossbar lever 32 generally resembles the shape of the alphabetical letter H. The base, bridge and transverse portions 42, 44 and 46 define both an upward facing U-shaped opening 48 and a downward facing U-shaped opening 50.

The surgeon holds the surgical tool 20 by placing the handgrip 34 in the palm of his or her hand with the thumb 52 extending around the rear of the handgrip 34 and resting against a flange 54 formed in a housing 56 of the handle assembly 22. The flange 54 is located vertically adjacent to the handgrip 34. A squeezing force applied by the thumb 52 toward the first finger 58 retains the handgrip 34 and thus the surgical tool 20 in the surgeon's hand. The flange 54 contacts the thumb 52 and the first finger joint to add further control and stability over the surgical tool.

The first, second, third and fourth fingers 58, 60, 62 and 64, respectively, extend forward from the palm of the hand and contact the crossbar lever 32. The second and third fingers 60 and 62 extend on opposite sides of the bridge portion 46. The first and second fingers are located in the upward facing U-shaped opening 48, and the third and fourth fingers are located in the downward facing U-shaped opening 50.

The bridge portion 46 allows the surgeon to continually squeeze the second and third fingers together against on the crossbar lever 32, and thereby establish an affirmative retention point on the lever to create substantial stabilization and control over the surgical tool 20, even when the fingers 58-64 are not firmly squeezed around the handgrip 34. The first and fourth fingers 58 and 64 also contribute to gripping the bridge portion 46 by pushing against the second and third fingers 60 and 62, respectively.

The additional control and stability provided by the continual gripping pressure against the bridge portion 46 is a substantial improvement over the prior art oval-shaped levers, were all four fingers are inserted into the oval opening of the lever. No part of the prior art oval-shaped lever allows affirmative retention force to be applied, because all of the oval-shaped opening surrounds the surgeon's fingers. The surgeon has the capability to pull the oval-shaped lever toward the handgrip by squeezing, or to push the oval-shaped lever outward by opening the fingers, or possibly to move the hand up and down within the oval-shaped lever opening, but none of these movements allow the surgeon to actually grip the lever in an affirmative way to help establish stability and control. In contrast, the ability to achieve an affirmative grip on the crossbar lever 32 by gripping the bridge portion 46 between the second and third fingers 60 and 62 allows the lever to be affirmatively held to contribute to stability and control.

The stability and control achieved by gripping the bridge portion 46 between the second and third fingers 60 and 62 extends over the entire range of pivoting movement of the crossbar lever 32. When the crossbar lever 32 is squeezed by the fingers 58-64 to a position adjacent to the handgrip 34 to close the jaws 28 and 30 around the tissue 40 as shown in FIG. 2, the fingers 60 and 62 are still capable of grasping the bridge portion 46. Usually stability and control is not an issue when the lever is squeezed against the handgrip, simply because the act of squeezing creates sufficient force to stabilize and control the surgical tool. When the crossbar lever 32 is pushed forward by the fingers 58-64 to an outward pivoted position away from the handgrip 34 to open the jaws 28 and 30 out of contact with the tissue 40 as shown in FIG. 4, the fingers 60 and 62 are still capable of grasping the bridge portion 46. The outward position shown in FIG. 4 is usually the most unstable and uncontrollable position, because moving the lever outward from the handgrip is intended to release all pressure on the lever, and the reduced pressure on the lever contributes to instability and lack of control since the only retaining force is a gripping force between the thumb and the first finger. The tendency for instability and lack of control is overcome by the attention pressure from fingers 60 and 62 against the bridge portion 46.

Should the jaws 28 and 30 resist opening, the surgeon applies a forward force to the transverse portion 44 by opening the fingers 58-64 to overcome the opening resistance of the jaws. The forward force is applied through all four fingers 58-64 while maintaining a grip on the bridge portion 46 of the crossbar lever 32 by squeezing pressure between the second and third fingers 60 and 62.

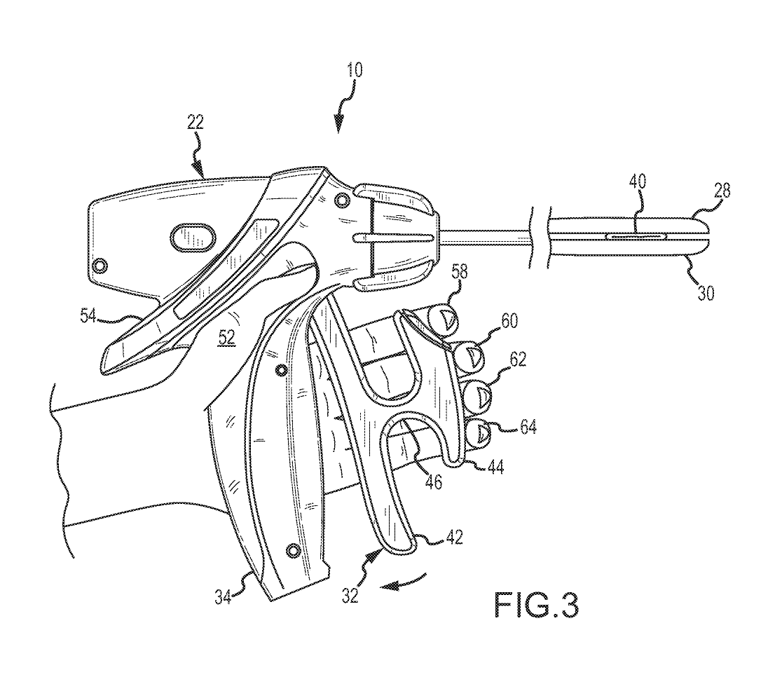

If additional gripping pressure is needed to pivot the crossbar lever 32 adjacent to the handgrip 34 to close the jaws, the fingers 58-64 may be extended to the outside of the transverse portion 44 as shown in FIG. 3. With the fingers on the outside of the transverse portion 44, greater advantage for squeezing force is achieved. In this regard, the crossbar lever 32 also obtains one of the benefits of the prior art oval-shaped lever which allows the surgeon to extend all four fingers to the outside of the oval shaped portion of the lever to achieve a greater squeezing advantage.

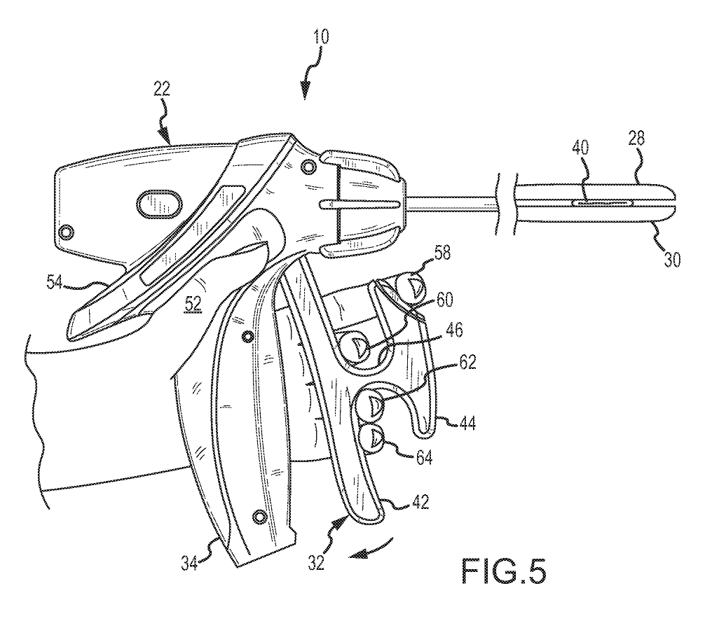

Another significant advantage of the crossbar lever 32 is that a control button switch 66 can be conveniently located on the transverse portion 44, as shown in FIG. 5. The control switch 66 is easily accessed by the first finger 58 when the surgeon manipulates the surgical tool and under any condition of squeezing or release of the crossbar lever 32. The control switch 66 is easily contacted with the first finger 58 to achieve various control functions, such as the application of energy to the tissue, as is generally understood from the second and third U.S. patent applications referenced above, Ser. Nos. (24.376) and (24.377). The affirmative retention by squeezing the bridge portion 46 between the second and third fingers 60 and 62 is maintained while the first finger 58 is removed to contact the control switch 66. The control switch 66 can be contacted by the first finger 58 in any position of the crossbar lever 62, as understood from FIGS. 2-5. Additional control switches can be incorporated in the transverse portion 44 at other locations, such as the bottom end (as shown), so that it may be accessed by the fourth finger 62. Locating an additional switch in this position still permits the second and third fingers to grip the bridge portion 46.

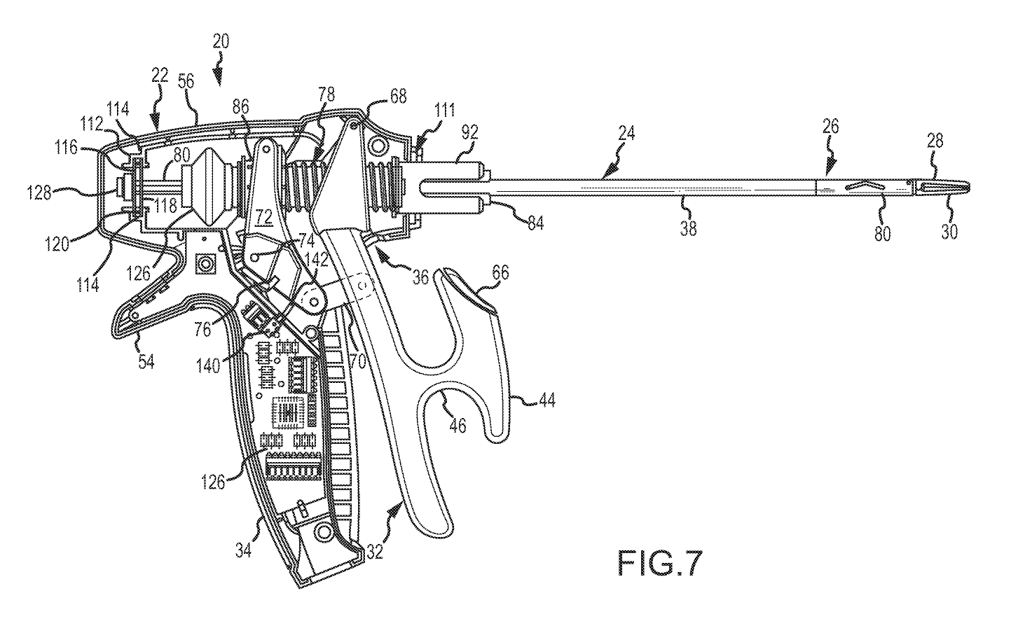

The crossbar lever 32 is one of the components of the operating mechanism 36 which transfers mechanical force from the crossbar lever 32 to the jaws 28 and 30, as is discussed below with reference to FIGS. 6-8. The crossbar lever 32 is pivotally attached to the housing 56 of the handle assembly 22 by a lever pivot pin 68. The crossbar lever 32 pivots about the pivot pin 68 as the crossbar lever 32 moves between the forward position shown in FIG. 6 and the squeezed position shown in FIG. 7. Mechanical force from the crossbar lever 32 is transferred through a rocker link 70 to a rocker arm 72. The rocker link 70 is pivotally connected to both the crossbar lever 32 and the rocker arm 72. The rocker arm 72 is pivotally attached to the housing at a rocker pivot pin 74. The rocker arm 72 pivots clockwise (as shown in FIGS. 6-8) about the rocker pivot pin 74 as the crossbar lever 32 is moved towards the handgrip 34. Conversely, the rocker arm 72 pivots counterclockwise (as shown) about the rocker pivot pin 74 as the crossbar lever 32 is moved away from the handgrip 34.

A bias spring 76 is wound around the rocker pivot pin 74 and contacts both the rocker arm 72 and the housing 56. The bias spring 76 biases the rocker arm 72 in the clockwise direction (as shown) about the rocker pivot pin 74. Thus, the bias spring 76 biases the crossbar lever 32 and the jaws 28 and 30 toward their open positions. Biasing the jaws 28 and 30 in the open position is advantageous because it generally gives the surgeon knowledge of the position of the jaws 28 and 30 in the absence of force applied to the crossbar lever 32.

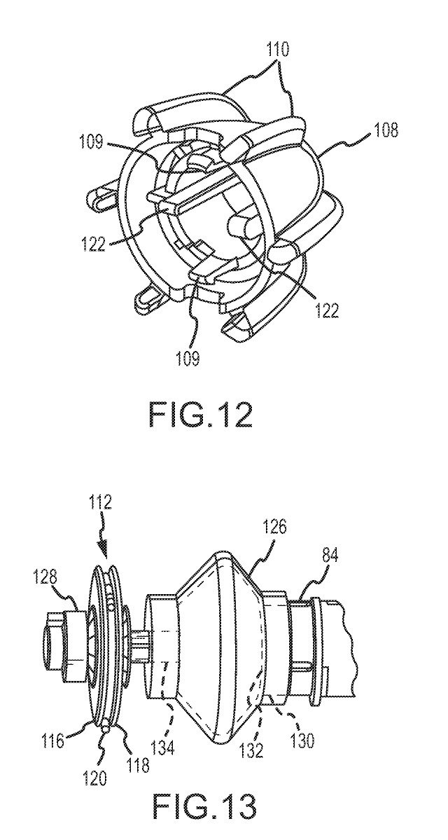

The rocker arm 72 is pivotally connected to a force limitation mechanism 78. The force limitation mechanism 78 prevents the application of an excessive amount of force from the crossbar lever 32 to the jaw movement mechanism 26. Excessive force applied to the jaw movement mechanism 26 might damage the jaw movement mechanism 26. Pivotal movement of the rocker arm 72 causes the force limitation mechanism 78 to create relative longitudinal reciprocating movement of the sleeve housing 38 with respect to a stationary rod 80. The sleeve housing 38 is formed with a hollow interior 82 (FIG. 12) within which a stationary rod 80 is mounted for relative longitudinal reciprocating movement with respect to the sleeve housing 38. The hollow interior 82 has a cross-sectional shape which is similar to the cross-sectional shape of the stationary rod 80 to prevent relative rotation of the sleeve housing 38 and the stationary rod 80.

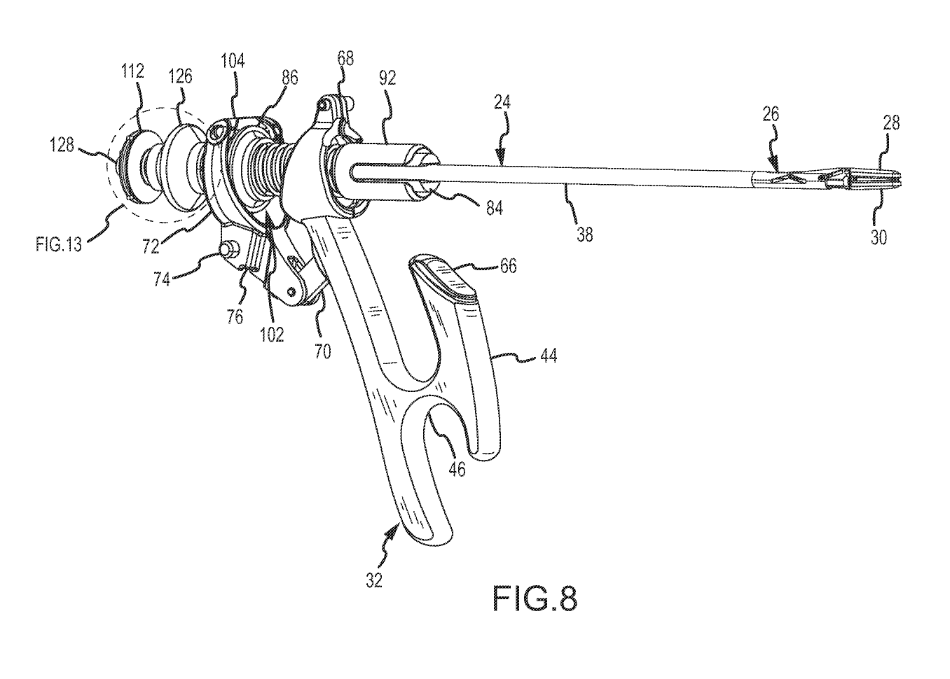

The force limitation mechanism 78 comprises a spool 84, a rocker adapter 86, a force limiting spring 88, a C-clip 90, and an adjustment cap 92, as shown in FIGS. 8-11. Both the rocker adapter 86 and the force limiting spring 88 define cylindrical openings which are slightly larger than the diameter of a cylindrical body 94 of the spool 84. The spool 84 defines an interior cylindrical opening which matches the outside diameter of the sleeve housing 38 (FIG. 8). The spool 84 is welded or otherwise affixed to the sleeve housing 38 (FIG. 8) so that the sleeve housing 38 moves in unison with the spool 84. The C-clip 90 fits within a groove 96 near the rear of the spool 84 and limits the rearward movement of the rocker adapter 86. Exterior threads 98 (FIG. 11) on a front portion of the spool 84 mate with interior threads 100 (FIG. 12) on a rear portion of the adjustment cap 92.

The force limitation mechanism 78 is assembled by fitting the C-clip 90 into the groove 96; sliding the rocker adapter 86 over the cylindrical body 94 of the spool 84; sliding the force limiting spring 88 over the cylindrical body 94; and screwing the adjustment cap 92 onto the spool 84 to compress the force limiting spring 88 to a desired precompression force. When precompressed over the spool 84, the force limiting spring 88 exerts a forward force against the adjustment cap 94 and a rearward force against the rocker adapter 86, which is prevented from moving rearward on the spool 84 by the C-clip 90. The rocker adapter 86 can move forward relative to the spool 84 only by further compressing the force limiting spring 88.

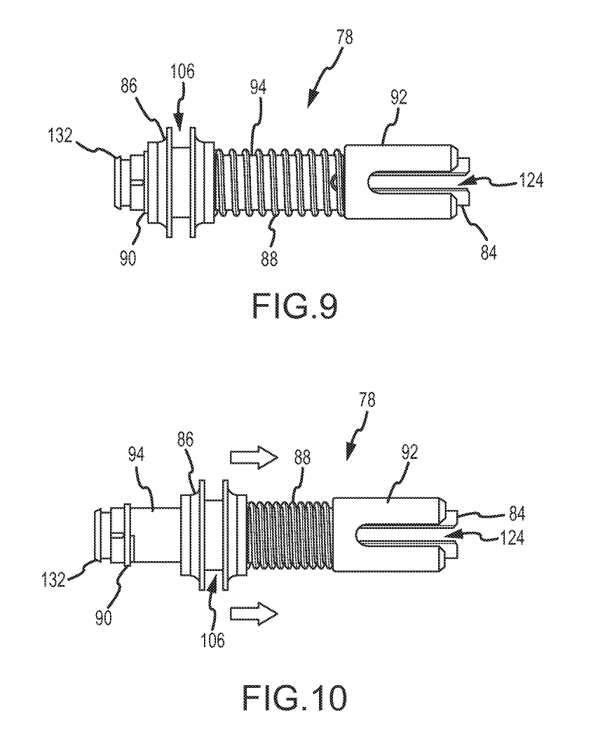

The rocker arm 72 defines a U-shaped opening 102 through which the force limitation mechanism 78 is mounted, as shown in FIG. 8. A rocker bridge 104 of the rocker arm 72 is positioned above the U-shaped opening 102 and encloses the top portion of the U-shaped opening 102. The rocker bridge 104 fits within a groove 106 (FIGS. 9 and 10) of the rocker adapter 86. The rocker bridge 104 stays within the groove 106 as the rocker arm 72 pivots. Mechanical force is transferred from the rocker arm 72 to the force limiting mechanism 78 through the rocker bridge 104 and to the rocker adapter 86. When the rocker arm 72 pivots clockwise (as shown in FIGS. 7 and 8), force is transmitted from the rocker bridge 104 to the rocker adapter 86 on a front side of the groove 106. Likewise, when the rocker arm 72 pivots counter clockwise, force is transmitted from the rocker bridge 104 to the rocker adapter 86 on a rear side of the groove 106.

In the absence of a rearward force on the crossbar lever 32, the force limiting spring 88 presses the rocker adapter 86 against the C-clip 90 with an amount of force related to the amount by which the force limiting spring 88 is precompressed. The force by which the force limiting spring 88 presses against the rocker adapter 86 in the precompressed state is referred to herein as the precompressed force, and is shown in FIG. 9. When the jaws 28 and 30 are in the closed position or when tissue 40 is compressed between the jaws 28 and 30, the jaws 28 and 30 will resist closing further. This resistance to further closing results in the sleeve housing 38 and the spool 84 resisting further forward movement relative to the stationary rod 80. So long as the force transferred from the rocker arm 72 to the rocker adapter 86 is less than the precompressed force, the rocker adapter 86 stays pressed against the C-clip 90 as shown in FIG. 9. When the force transferred from the rocker arm 72 to the rocker adapter 86 exceeds the precompressed force, the rocker adapter 86 slides forward relative to the spool 84 and further compresses the force limiting spring 88 as shown in FIG. 10. The force limiting spring 88 therefore absorbs the additional force without transferring it through the sleeve housing 38 to the jaw movement mechanism 26. The force limiting mechanism 78 thus prevents a potentially damaging level of mechanical force from reaching the jaw movement mechanism 26.

The jaws 28 and 30 are in the open position to capture tissue 40 between the jaws 28 and 30. Prior to capturing tissue, the jaws 28 and 30 are typically aligned so that the plane of movement of the jaws 28 and 30 is perpendicular or transverse to the tissue to be captured. Positioning the jaws 28 and 30 in alignment with tissue to be captured is achieved by rotating the entire surgical tool 20 including the handle assembly 22, and may result in the handgrip 34 extending in an awkward position for the surgeon.

To allow the surgeon a greater degree of flexibility in positioning the surgical tool 20, the shaft assembly 24, including the jaw movement mechanism 26, the jaws 28 and 30 and the force limiting mechanism 78, are rotatable with respect to the housing 56 of the handle assembly 22. The rotation occurs about a longitudinal axis of the shaft assembly 24. The shaft assembly 24 is rotated relative to the handle assembly 22 by rotating a spinner 108 relative to the housing 56, as shown in FIGS. 1-5 and 12. Knurls 110 are axially spaced around a circumference of the spinner 108 to better allow the surgeon to grasp and turn the spinner 108. Hook clips 109 (FIG. 12) attached to the spinner 108 snap into a circular recess 111 (FIGS. 6 and 7) of the housing 56. The hook clips 109 secure the spinner 108 in place with respect to the housing 56 while allowing the spinner 108 to rotate about the sleeve housing 38.

The rotation of the shaft assembly 24, jaw movement mechanism 26 and jaws 28 and 30 relative to the handle assembly 22 are described below with reference to FIGS. 6-8 and 11-13. The rear end of the stationary rod 80 is attached to a dual clutch device 112 (FIG. 13) which is compressed within a clutch recess 114 (FIGS. 7 and 8) formed into the housing 56. The dual clutch device 112 includes two discs 116 and 118 (FIG. 13) which are pressed apart by a clutch clip 120 (FIG. 13). The two discs 116 and 118 are fixedly attached to the stationary rod 80 so that the discs 116 and 118 rotate with rotation of the rod 80.

The dual clutch device 112 prevents longitudinal movement of the stationary rod 80 due to being confined and compressed within the clutch recess 114. When the dual clutch device 112 is compressed within the clutch recess 114, the clutch clip 120 pushes the discs 116 and 118 apart so that the discs 116 and 118 press against sides of the clutch recess 114. The clutch discs 116 and 118 have friction surfaces which resist rotation within the clutch recess 114.

Inner protrusions 122 (FIG. 12) of the spinner 108 fit within slots 124 (FIG. 11) of the spool 84 and the adjustment cap 92 and cause the spinner 108 and the spool 84 to rotate in unison. The slots 124 of the adjustment cap 92 and the spool 84 are aligned as shown in FIGS. 9 and 10 so that the inner protrusions 122 of the spinner 108 fit within both sets of slots 124. Rotational movement of the spinner 108 is transferred to the spool 84 through the inner protrusions 122 and the slots 124. Rotational movement of the spool 84 is transferred to the sleeve housing 38 due to the spool 84 being welded or otherwise fixedly attached to the sleeve housing 38. Rotational movement of the sleeve housing 38 is transferred to the stationary rod 80 due to the non-circular cross-sectional shape of the hollow interior 82 of the sleeve housing 38 matching the cross-sectional shape of the stationary rod 80. Finally, rotation of the stationary rod 80 is allowed but partially resisted by the dual clutch device 112. The resistance to rotation of the dual clutch device 112 is enough so that the stationary rod 80 does not inadvertently rotate yet not so much as to hinder a desired rotation of the shaft assembly 24 (FIG. 1) by the surgeon.

During laparoscopic surgical procedures, the forward end of the surgical tool 20 including the jaws 28 and 30 and the jaw movement mechanism are inserted into a pressurized body cavity. Seals 126 and 128 prevent pressurized gas from within the cavity from escaping through the shaft assembly 24. Seal 126 (FIG. 13) has a forward opening 130 which mates with a flared end 132 of the spool 84. The seal 126 has a rearward opening 134 which defines a cross-sectional shape the same as the stationary rod 80. Any pressurized gas which may flow from the cavity at the surgical site between the sleeve housing 38 and the stationary rod 80 is contained within the seal 126 and not allowed to escape into the housing 56 of the surgical tool 20.

The stationary rod 80 is hollow and contains conductive wires (not shown) which conduct electrical energy to heating elements (not shown) of the jaws 28 and 30. The seal 128 (FIGS. 6 and 7) is affixed to the rearmost portion of the stationary rod 80 and prevents any pressurized gas within the stationary rod 80 from leaking from the pressurized body cavity into the housing 56 of the surgical tool 20. The conductive wires exit the stationary rod 80 through small holes (not shown) within the seal 128.

Certain operational features of the surgical tool 20 are described above in connection with the second and third above-referenced U.S. patent application Ser. Nos. (24.376) and (24.377). In addition, FIGS. 6 and 7 show a circuit board 136 is positioned within the housing 56 of the handle assembly 22. The circuit board 136 contains the electronic components which control the electrical operation of the surgical tool 20, and which interact with electronic components of a power source (not shown) which is connected to the surgical tool 22 deliver electrical energy to heating elements integrated in the jaws 28 and 30.

Different conditions of use of the surgical tool 20 are accomplished by operating the control switch 66 on the crossbar lever 32 (FIGS. 1-8) and one or more side switches 138 located on the flange 54 of the housing 56 of the handle assembly 22 (FIGS. 1-5). Both the control switch 66 and the side switches 138 are electrically connected to the circuit board 136 through conductors (not shown). The control switch 66 is positioned at a front forward location on the crossbar transverse portion 44. The control switch 66 is within reach of the first finger 58 (FIGS. 2-5). The side switches 138 are located on the flange 54 on both sides of the housing 56. The side switches 138 are within reach of the surgeon's thumb while holding the surgical tool 20. Both side switches 138 are ideally operative to perform the same surgical procedure. This allows the surgeon to select the desired surgical procedure indicated by the side switches 138 with the thumb of either the right or left hand holding the surgical tool 20.

The circuit board 136 includes conventional electronic components which are organized and connected and/or programmed to associate different types of surgical procedures to the control switch 66 and the side switches 138 depending on the intended procedure to be performed with of the surgical tool 20. For example in the case where the surgical tool 20 is a tissue sealing surgical tool, the control switch 66 might be associated with a tissue seal procedure in which tissue is only sealed and the side switches 138 might be associated with a combined cut and seal procedure in which tissue is first sealed and then cut by the continued application of energy to the tissue.

An arming switch 140 is closed by a rocker arm projection 142 when the crossbar lever 32 is moved rearward by a predetermined amount, as shown in FIGS. 6 and 7. The arming switch 140 is mounted on and electrically connected to the circuit board 136. The rocker arm projection 142 (FIGS. 6-8) extends from beneath the rocker arm 72 at a position to the rear of the rocker pivot pin 74. As the crossbar lever 32 is moved rearward, the rocker arm 72 pivots clockwise (as shown) moving the rocker arm projection 142 towards the arming switch 140 and eventually closing the arming switch 140. The arming switch 140 is thus open when the crossbar lever 32 is in the open position, and is closed when the crossbar lever 32 is in a partially or fully squeezed position. The arming switch 140 prevents energy delivery to the jaws 28 and 30 unless the crossbar lever 32 is squeezed enough to close the arming switch 140. The arming switch 140 prevents the accidental initiation of a surgical procedure of the surgical tool 20 when the jaws 28 and 30 are in the fully opened position.

The present invention allows a surgeon to establish and maintain better control and stability over the surgical tool 20 while it is used, and particularly when the lever is in the opened position. The bridge portion 46 of the crossbar lever 32 allows the surgeon to apply a positive gripping retention on the lever to achieve this better control and stability. The transverse portion 44 of the crossbar lever 32 allows additional gripping force to be applied by grasping the outside of the transverse portion 44 with the fingers, if desired. The transverse portion 44 of the crossbar lever 32 also permits at least one control switch 66 to be located for convenient access by one of the fingers when squeezing or otherwise manipulating the crossbar lever 32.

In addition, the rocker link 70 and the rocker arm 72 transfer force from the pivoting crossbar lever 32 to move the jaws with greater efficiency due to the geometric arrangement and pivot points of the crossbar lever 32 and the rocker arm 72. The force limitation mechanism 78 of the surgical tool 20 prevents excessive force from being transferred from the crossbar lever to the jaws of the surgical tool, thereby preventing excessive and potentially damaging force from reaching the jaw movement mechanism 26 and the jaws 28 and 30. The rotational capability of the shaft assembly 24, including the jaw movement mechanism 26 and the jaws 28 and 30 affords the surgeon additional flexibility in maneuvering the jaws 28 and 30 and the tool 20 to capture tissue between the jaws.

Many other advantages and improvements will be apparent upon fully appreciating the various aspects of the present invention. Presently preferred embodiments of the present invention and many of its improvements have been described with a degree of particularity. The above description is a preferred example of implementing the invention, but that exemplary description is not necessarily intended to limit the scope of the invention. The scope of the invention is defined by the following claims.

What is claimed:

1. In a surgical tool having a handle assembly which includes a handgrip and a lever which pivots relative to the handgrip in response to squeezing movement from fingers of a user's hand that also contacts the handgrip, an elongated shaft assembly connected to and extending from the handle assembly, tissue contact elements connected to a forward end of the shaft assembly for manipulating tissue, and an operating mechanism within the handle assembly for transferring pivotal movement of the lever relative to the handgrip into motion which moves the tissue contact elements, and an improvement to the lever comprising:
a crossbar lever having a base portion and a bridge portion and a transverse portion configured generally in an H-shape, the transverse portion located forward of the base portion and extending generally in alignment with the base portion, the bridge portion extending from a general midpoint of the transverse portion rearward to the base portion, the H-shape configuration of the base, bridge and transverse portions defining an upward facing U-shaped opening for receiving first and second fingers of the user's hand and a downward facing U-shaped opening for receiving third and fourth fingers of the user's hand, the bridge portion extending between the second and third fingers of the user's hand and receiving squeezing force from the second and third fingers to stabilize and control the surgical tool in the hand of the user.

2. A surgical tool as defined in claim 1, wherein:
one of the base portion or the transverse portion receiving squeezing force from the fingers to pivot the crossbar lever toward the handgrip.

3. A surgical tool as defined in claim 2, further comprising:
a control element connected to the transverse portion at a location to be contacted by one of the fingers, the control element controlling an aspect of surgical operation of the tool.

4. A surgical tool as defined in claim 3, wherein:
the control element comprises an electrical switch.

5. A surgical tool as defined in claim 4, wherein:
the switch is positioned on the transverse portion at a location to be contacted by one of the fingers while the other fingers squeeze the one of the base portion or the transverse portion.

6. A surgical tool as defined in claim 5, wherein:
the switch is positioned on the transverse portion at a location to be contacted by a first one of the fingers.

7. A surgical tool as defined in claim 1, wherein:
the shaft assembly comprises a pair of longitudinally relatively movable reciprocating shaft members;
the operating mechanism moves the shaft members with longitudinal reciprocating motion in response to pivoting movement of the crossbar lever;
a movement mechanism is connected to the shaft members at a forward end of the shaft assembly and is operative for converting the longitudinal reciprocating motion of the shaft members into movement of the tissue contact elements; and
the operating mechanism further comprises a rocker arm connected with the crossbar lever to pivot in response to pivoting movement of the crossbar lever, and an adapter connected to one of the shaft members and contacted by the rocker arm to translate the pivoting movement of the rocker arm to one of the shaft members as longitudinal reciprocating motion of one of the shaft members.

8. A surgical tool as defined in claim 7, wherein:
the operating mechanism further comprises a link member pivotally connecting the crossbar lever to the rocker arm to transfer force from the crossbar lever to the rocker arm as the crossbar lever is pivoted.

9. A surgical tool as defined in claim 7, wherein:
the tissue contact elements include jaws which are movable toward or away from one another to compress tissue therebetween.

10. A surgical tool as defined in claim 9, wherein:
the operating mechanism further comprises a force transfer mechanism connected between the adapter and the one of the shaft members, the force transfer mechanism including a force limiting element for limiting the amount of force transferred between the adapter and the one shaft member.

11. A surgical tool as defined in claim 10, wherein:
the force transfer mechanism further includes a cap member connected to the one shaft member;
the force limiting element comprises a spring which surrounds the one shaft member and extends between the adapter and the cap member.

12. A surgical tool as defined in claim 10, wherein:
the spring is precompressed to a predetermined extent between the adapter and the cap member; and
the spring accepts additional force beyond the predetermined extent of precompression arising from force transferred from the rocker arm to the adapter upon pivoting of the crossbar lever.

13. A surgical tool as defined in claim 12, wherein:
the force transfer mechanism further includes a spool which defines the cap member at a forward end and around which the spring is positioned; and
the adapter is positioned around the spool.

14. A surgical tool as defined in claim 10, wherein:
annular openings are defined in both the rocker arm and the crossbar lever;
the force transfer mechanism and the one shaft member extend through the annular openings of both the rocker arm and the crossbar lever;
the shaft assembly is selectively rotatable about its longitudinal axis and relative to the handle assembly; and
the force transfer mechanism and the one shaft member are rotatable with the shaft assembly within the annular openings of both the rocker arm and the crossbar lever.

15. A surgical tool as defined in claim 1, wherein:
the handle assembly comprises a flange extending on opposite lateral sides and around the rear portion of the handgrip at a location vertically adjacent to the handgrip, the flange is positioned for contact by a thumb and a joint of the first finger to contribute stabilization and control of the surgical tool.

16. A surgical tool as defined in claim 15, further comprising:
a control element connected to the flange at a location to be contacted by the thumb, the control element controlling an aspect of surgical operation of the tool.

17. A surgical tool as defined in claim 16, wherein:
the control element comprises an electrical switch.

18. In a surgical tool having a handle assembly which includes a handgrip and a lever which pivots relative to the handgrip in response to squeezing movement from fingers of a user's hand that also contacts the handgrip, an elongated shaft assembly connected to and extending from the handle assembly, the shaft assembly including relative longitudinal movement shaft members, jaws connected to a forward end of the shaft assembly for manipulating tissue, and an operating mechanism within the handle assembly for transferring pivotal movement of the lever relative to the handgrip into motion which moves the jaws, and an improvement to the operating mechanism comprising:
a rocker arm connected with the lever to pivot in response to pivoting movement of the lever;
an adapter connected to one of the shaft members and contacted by the rocker arm to transfer pivoting movement of the rocker arm through the adapter to one of the shaft members as longitudinally reciprocating motion;
a link member pivotally connecting the crossbar lever to the rocker arm to transfer force from the crossbar lever to the rocker arm as the crossbar lever is pivoted;
a force transfer mechanism connected between the adapter and the one of the shaft members, the force transfer mechanism including a cap member connected to the one shaft member and a spring which surrounds the one shaft member and which extends between the adapter and the cap member, the spring limiting the amount of force transferred between the adapter and the one shaft member.

19. A surgical tool as defined in claim 18, wherein:
the spring is precompressed to a predetermined extent between the adapter and the cap member; and
the spring accepts additional force beyond the predetermined extent of precompression arising from force transferred from the rocker arm to the adapter upon pivoting of the crossbar lever.

20. A surgical tool as defined in claim 18, wherein:
annular openings are defined in both the rocker arm and the crossbar lever;
the force transfer mechanism and the one shaft member extend through the annular openings of both the rocker arm and the crossbar lever;
the shaft assembly is selectively rotatable about its longitudinal axis and relative to the handle assembly; and
the force transfer mechanism and the one shaft member are rotatable with the shaft assembly within the annular openings of both the rocker arm and the crossbar lever.
